(19) United States
(12) Patent Application Publication
(10) Pub. No.: US 2012/0283556 A1
(43) Pub. Date: Nov. 8, 2012

(54) Method for assisting optimum positioning of an occlusion site in a blood vessel in a tumor embolization

(76) Inventors: Sigrid Ferschel, Uttenreuth (DE); Stefan Lautenschläger, Hausen (DE)

(21) Appl. No.: 13/463,839

(22) Filed: May 4, 2012

(30) Foreign Application Priority Data
May 6, 2011 (DE) 102011075419.9

(51) Int. Cl. A61B 6/00; A61B 5/055; A61B 6/03

(52) U.S. Cl. 600/427

(57) ABSTRACT

A method for assisting optimum positioning of an occlusion site in a blood vessel in a tumor embolization is provided. Tumors are cut off from the blood supply by the embolization which is an artificial occlusion of blood vessels. The vessels around the tumor and at the same time the planned site for occlusion are firstly determined. A path from the access to the vessels can be determined based on image recognition, and with the aid of this path the site for occlusion can then be optimally determined. A computer system assists the doctor carrying out the treatment by a suitable display.

### CROSS REFERENCE TO RELATED APPLICATIONS

This application claims priority of German application No. 10 2011 075 419.9 filed May 6, 2011, which is incorporated by reference herein in its entirety.

### FIELD OF INVENTION

The invention relates to the field of assisting a specific medical intervention, namely what is referred to as embolization.

### BACKGROUND OF INVENTION

Embolization takes place in what is known as vascular tumors, such as tumors in the liver, the lungs, etc. Embolization is the artificial occlusion of blood vessels by administration of liquid plastics, plastic spheres, fibrin sponges or the like via a catheter. Embolization is conventionally carried out using radiographic imaging, i.e. with the aid of 2D X-ray images (fluoroscopic images). Due to this occlusion of the blood vessels the tumor is cut off from the blood and nutrient supply and dies off.

It is essential when carrying out the embolization that it is ensured that all supplying vessels are occluded because it is only in this way that the tumor is reliably destroyed. Searching for and embolizing the supplying vessels leads to high radiation exposure for the patient and doctor carrying out the treatment owing to the necessary radiographic imaging. Digital subtraction angiography images (2D DSA images) are typically used: The patient is imaged once without and a second time with contrast medium in his body and the two images are subtracted from each other so the vessels, which convey the contrast medium, can be seen particularly clearly. This method is, moreover, also very expensive, in other words time-consuming and therewith also costly. The doctor also has to ensure that only those vessels are occluded which lead into the tumor since otherwise he would destroy other tissue.

### SUMMARY OF INVENTION

It is the object of the invention to disclose a method with the aid of which the location of an occlusion—i.e. the location where embolization takes place—can be optimally placed (positioned) so during subsequent treatment the tumor is cut off from the blood supply and other tissue is spared.

The object is achieved by a method with the features as claimed in the claims.

In the case of the inventive method a 3D image data record relating to the patient is obtained. Blood vessels surrounding the tumor are identified with the aid of the 3D image data record on the one hand and on the other hand a site of an occlusion device (e.g. of a catheter) in the coordinate system is determined which is associated with the 3D image data record. Paths running in blood vessels from this site of the occlusion device to the blood vessels surrounding the tumor are then determined by image recognition with the aid of the 3D image data record. With the aid of the paths it is then checked whether the site of the occlusion device satisfies at least one predetermined criterion (for instance whether all blood vessels surrounding the tumor are reached starting from the site of the occlusion device, and/or whether no other sites are reached). If this is not the case, i.e. the predetermined criterion is not satisfied, a new site is calculated for the occlusion device (and optionally communicated to an operator in any medium, visually, acoustically or haptically).

The invention is based on the recognition that methods for determining such paths through blood vessels which are available anyway—for example as established methods by the name of “region growing”—may also be used in the present case. A 3D image data record is very significant here, and when it is obtained allows the paths to be determined. Since in the present case there is a connecting problem, namely whether starting from the site of the occlusion device, i.e. the planned location for the occlusion, all blood vessels surrounding the tumor are reached, but not any others, these paths are helpful in determining the site for the occlusion device. The optionally automatic calculation of a new site frees the doctor carrying out the treatment from being compelled to act himself and perform examinations.

In a preferred embodiment of the invention branches in the blood vessels that occur along the paths are examined as to whether they lead to a blood vessel surrounding the tumor (and this is the same as one path branching from the other path), and if this is not the case the site for the occlusion device is moved along one of the paths (namely the one from which the other paths branch off), until all blood vessels branching from the path are also part of one path, i.e. all lead to a blood vessel (optionally other blood vessels respectively) surrounding the tumor.

The invention is based on the recognition that there are certain cord-like structures in blood vessels, so as a rule the identification of a single occlusion site and occlusion of the blood vessel is sufficient there to reliably cut off the tumor from the blood.

The site of the occlusion device can be determined with the aid of different methods: It is particularly simple if a user input relating to a display obtained from the 3D image data record is received. In other words, the doctor carrying out the treatment can simply mark a blood vessel in a corresponding display (e.g. a 2D projection or a section) with the aid of a positioning device, such as a mouse or another human-machine interface, at the point where he plans the occlusion. The site of the occlusion device is therefore technologically available due to receipt of the user input.

The site of the occlusion device can alternatively be determined by way of image recognition: In this case the doctor carrying out the treatment guides the occlusion device, such as e.g. the catheter, to the location at which he would plan the occlusion, i.e. the embolization, per se. The catheter is located by image recognition and it is then checked by way of the inventive method merely whether the doctor has found a good location therewith, and he is optionally informed about a different site for occlusion device.

To identify the blood vessels surrounding the tumor it is helpful if the tumor is identified first of all. This can occur in particular by way of the step of what is known as segmenting of the 3D image data record, if, in other words, a specific gray value is allocated to groups of gray values, gray values from a gray value interval, and a gray value which is very different therefrom is allocated to other gray values; the tumor is then visible in the images in high contrast. Once the tumor has been identified the blood vessels in the vicinity of the tumor can then be identified by way of a further segmenting process (namely in relation to blood vessels). Such stepwise segmenting is known per se from the prior art.

Segmenting can take place completely automatically on the one hand, but on the other hand may also take place following receipt of a corresponding input, for example when a doctor carrying out the treatment clicks on a point in a display of the 3D image data record a gray value can be determined which corresponds to a mean gray value in the region of the tumor. The doctor can optionally also roughly sketch the contours of the tumor already, for example simply place a circle or a sphere in relation to the 3D image data record.

In a further preferred embodiment of the invention a 2D X-ray image data record relating to the patient is obtained. On the basis of an allocation of the 3D image data record an overlaid display comprising the 2D and 3D image data records is then prepared, wherein in this display the last fixed (i.e. last determined and optionally calculated) site for the occlusion device is marked in the display. In other words, an X-ray image in the form of a fluoroscopy image is placed in relation to the 3D image data record such that the doctor can identify the desired position for the occlusion device (i.e. the last fixed site) in the display on the one hand and on the other hand can accurately guide his occlusion device (the catheter) to this location using fluoroscopic imaging.

The 3D image data record can subsequently be obtained using a different device (image recording device) from the 2D X-ray image data record. In this case it is helpful if the 3D image data record relating to the 2D X-ray image data record is registered to allow the overlaid display. Registration includes the calculation and specification of a mapping rule, i.e. a positionally and dimensionally correct allocation of the coordinate systems which form the basis of the respective image recording devices and are therewith implicit to the respective image data records.

As an alternative to this the 3D image data record can be recorded using the same image recording device as the 2D X-ray image data record; registration can then be omitted.

### BRIEF DESCRIPTION OF THE DRAWINGS

A preferred embodiment of the invention will be described in more detail below with reference to the drawings, in which:

FIG. 1 shows a flowchart to illustrate this preferred embodiment of the inventive method; and

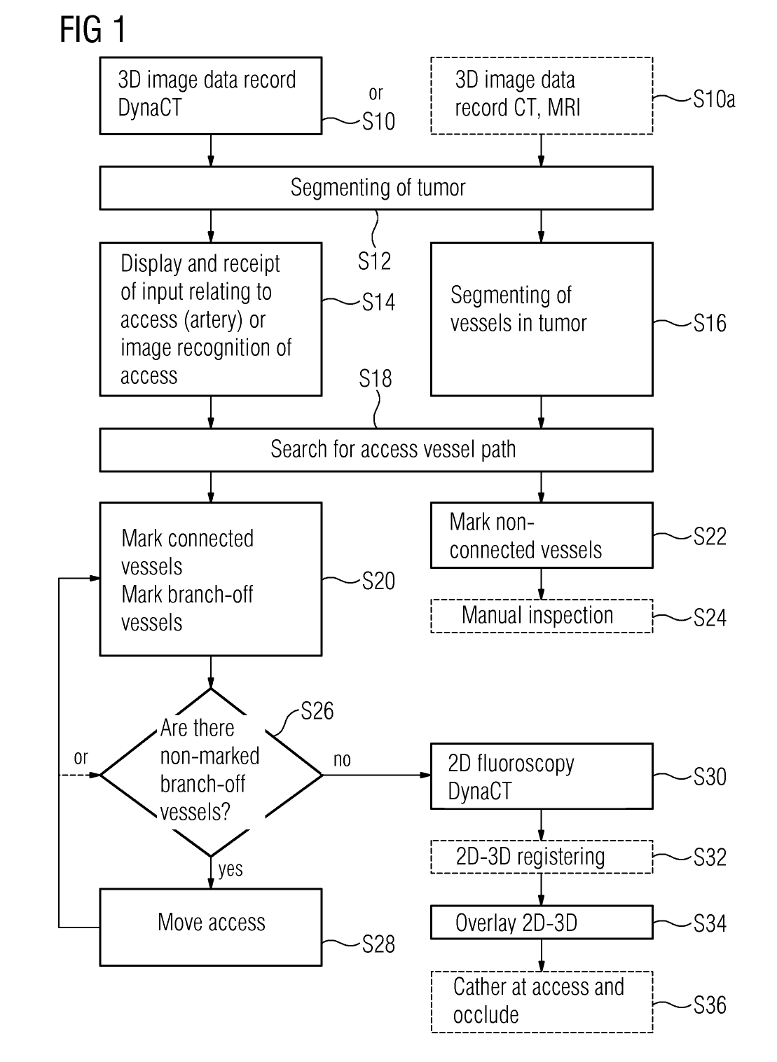

FIG. 2 shows a schematic diagram to illustrate the concept of the connecting path in a system of blood vessels with a tumor supplied by such blood vessels.

### DETAILED DESCRIPTION OF INVENTION

A patient shall in the present case be suffering from a vascular tumor, wherein by way of embolization the blood vessels supplying this tumor are to be cut off from the supply, and therewith the tumor as well so that it dies off.

The method begins in that first of all a 3D image data record of a patient is recorded in step S10. This can be done with the aid of what is known as the DynaCT® system from Siemens, i.e. an X-ray angiography device which can obtain a plurality of 2D image data records and from these, for example by way of filtered back projection, calculates a 3D X-ray image data record. Alternatively a 3D image data record can be obtained in step S10a with the aid of conventional computerized tomography or using magnetic resonance (MRI).

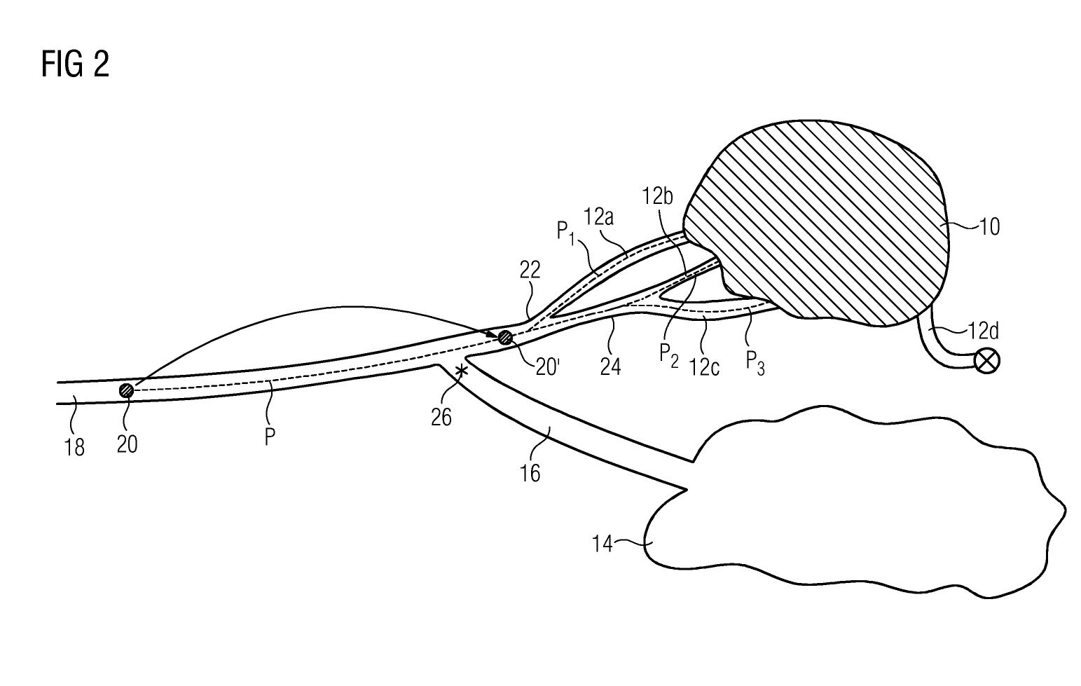

As a result a 3D image data record is made available in which for example the elements shown in FIG. 2 are depicted: A tumor 10 is supplied with blood by a plurality of blood vessels 12a, 12b, 12c, optionally also by a blood vessel 12d. In addition to the tumor 10 there is also an organ 14 which is also supplied with blood via a vessel 16. The blood supply originates as a whole from a blood cord 18 into which a catheter can be introduced to perform embolization.

It may be determined from the 3D image data record where the tumor 10 is located: This can occur by way of what is known as segmenting of the tumor in step S12. Segmenting is a method known per se which can also be applied to tumors. A specification can optionally be made for segmenting by way of a user input, for example it may be specified in which gray value interval gray values from the 3D image data record are to be interpreted as indicative of tumor tissue.

In step S14 a display of the 3D image data record with the segmented tumor is then provided for the doctor carrying out the treatment. The doctor then decides that embolization is carried out via the cord 18 and he chooses point 20 by way of example as the site for the supply of plastic spheres for the purpose of embolization. He can communicate this site 20, for example by way of an input at a mouse, to the computer system which processes the data and shows him the 3D image data record. Alternatively image recognition can occur if a catheter for occlusion of the blood vessel can already be seen in the 3D image data record, and this is possible in particular when carrying out step S10 (and less when carrying out alternative step S10a).

At the same time as step S14 or before or after, segmenting takes place independently in step S16 in relation to the vessels 12a, 12b and 12c around the tumor. It is a known measure, if a first area has initially been demarcated by segmenting, to carry out a further segmenting process in a neighboring region to identify structures therein. The computer system can thus identify the vessels 12a, 12b, 12c and 12d automatically.

A fundamental step now takes places as step S18 in the present method: A path P is sought from the access 20 to the vessels 12a, 12b, 12c. The path P splits into secondary paths P₁, P₂ and P₃.

In step S20 it is then possible for the computer system to mark the vessels connected to point 20, namely vessels 12a, 12b and 12c in the present case. All branch-off vessels, from which a path branches, are also marked, i.e. for example branch-off vessel 22, where path P₁ branches from path P, branch-off vessel 24, where paths P₂ and P₃ branch from path P, etc. Irrespective thereof vessels not connected to point 20 are marked in step S22, i.e. vessel 12d is marked in the present case, and, more precisely, in a different manner from vessels 12a, 12b and 12c. Vessel 12d is not connected to point 20 and must therefore be examined separately later by the doctor carrying out the treatment in a step S24. In the present case it is sufficient for the method at hand that these vessels are marked, the remainder are not the computer system's responsibility.

Following step S20 it is accordingly checked in step S26 whether there are non-marked branch-off vessels. This would be branch-off vessel 26 in the present case: A non-marked branch-off vessel is a branch-off vessel which does not lead to a region (organ) 14, connected to the vessels 12a, 12b, 12c in the vicinity of the tumor, which has nothing to do with the tumor. Nevertheless this organ 14 would be cut off from the blood supply if embolization was placed at point 20. For this reason the access point 20 is moved to point 20′ in step S28 and, more precisely, the point 20′ is sought along path P which is closer to the tumor 10 and does not cut off the blood supply for access 26. As a consequence steps S18, S20, S26 can then be repeated again or step S26 can follow directly if it is certain that the originally allocated paths and markings are reliably placed.

At some point there will be no more non-marked branch-off vessels behind the respectively determined, last-valid value for access site 20′.

A 2D radioscopy is then obtained in step S30 with the aid of the DynaCT® system from Siemens, i.e. a 2D X-ray image (fluoroscopy image). In step S34 this 2D display of the patient should be overlaid with the tumor 10 of the 3D display from the originally obtained 3D image data record. Simple overlaying is possible if step S10 has been carried out in advance. If the alternative according to S10a was selected, step S32 of registering the 3D image data record with the 2D image data record from S30 must also take place in the meantime, i.e. a 3D-2D registering, i.e. a positionally and dimensionally correct allocation of the image data and coordinate systems to each other.

The overlaid display according to step S34 means the doctor carrying out the treatment can accordingly identify both the current position of the catheter, for example with the aid of a marker on the catheter, and the tumor 10 (optionally emphasized by segmenting) with the supplying blood vessels 12a, 12b, 12c. The last-calculated access to the site 20′ is shown marked for the doctor, moreover, so the doctor knows to where he has to guide his catheter.

Outside of the method carried out by the computer system the catheter can then be guided in step S36 to the access and the actual occlusion can be performed by the doctor carrying out the treatment.

The invention is performed with the aid of devices, namely at least one image recording device, optionally two different image recording devices, and a data processing device (not shown). The doctor carrying out the treatment is assisted in his activity by the data processing device in that he is provided with information or graphic displays with the aid of which he can orientate himself. As a result of the fact that in step S28 access 20 is moved to access 20′ or the like respectively, the doctor carrying out the treatment is relieved of one decision by the computer system. However, these decisions only include geometric considerations, namely of the connection between the blood vessels 12a, 12b, 12c and point 20′ on the one hand and of the non-connection to region 14 on the other hand.

1. A method for assisting positioning an occlusion site in blood vessels for cutting off a tumor in a patient, comprising:
obtaining a 3D image data record of the patient;
identifying the blood vessels surrounding the tumor from the 3D image data record;
determining the occlusion site in a coordinate system of the 3D image data record;
determining paths running from the occlusion site to the blood vessels based on the 3D image data record by an image recognition;
checking whether the occlusion site satisfies a predetermined criterion based on the paths; and
determining a new occlusion site if the occlusion site does not satisfy the predetermined criterion.

2. The method as claimed in claim 1, wherein branches of the blood vessels that occur along the paths are examined to whether the branches lead to the blood vessels surrounding the tumor, and wherein the occlusion site is moved along the paths until the branches of the blood vessels are part of the paths.

3. The method as claimed in claim 1, wherein the occlusion site is determined with a user input by a display from the 3D image data record.

4. The method as claimed in claim 1, wherein the occlusion site is determined by the image recognition.

5. The method as claimed in claim 1, wherein the tumor is identified by segmenting the 3D image data record and the blood vessels surrounding the tumor are identified by a further segmentation.

6. The method as claimed in claim 1, wherein a 2D X-ray image data record of the patient is obtained and is overlaid displayed with the 3D image data record in which a last determined occlusion site is marked.

7. The method as claimed in claim 6, wherein the 3D image data record is registered with the 2D X-ray image data record.

8. The method as claimed in claim 6, wherein the 3D image data record is obtained using a same image recording device as the 2D X-ray image data record.
